(19) United States
(12) Patent Application Publication
(10) Pub. No.: US 2018/0049837 A1
(43) Pub. Date: Feb. 22, 2018

(54) SURGICAL PROBE CALIBRATION SYSTEM AND METHOD

(71) Applicant: UNIVERSITY HOSPITALS CASE MEDICAL CENTER, Cleveland, OH (US)

(72) Inventors: Adonis K. Hijaz, Westlake, OH (US); Colin K. Drummond, Lakewood, OH (US)

(21) Appl. No.: 15/557,250

(22) PCT Filed: Mar. 14, 2016
(86) PCT No.: PCT/US2016/022302

§ 371 Date: Sep. 11, 2017

Related U.S. Application Data
(60) Provisional application No. 62/132,737, filed on Mar. 13, 2015.

(51) Int. Cl. A61B 90/00; A61F 2/00

(52) U.S. Cl. CPC A61B 90/06; A61B 2017/00805; A61F 2/0045

(57) ABSTRACT

A smart probe for use during an implantation procedure to provide a desired spacing between the implanted device and a body part while measuring the pressure exerted by the implanted device on the body part to ensure that the implanted device incorporates a desired tension, and thereby a desired pressure, on the body part. Also, a procedure for using the smart probe during the implantation procedure is disclosed.

### CROSS-REFERENCES TO RELATED APPLICATIONS

This applications claims priority from U.S. Provisional Patent Application Ser. No. 62/132,737 filed on Mar. 13, 2015, and incorporated herein by reference.

### BACKGROUND

For the typical person, “bladder control” is a straight-forward task. Three critical muscle systems ordinarily work in concert (without too much thought by the individual during voiding) to control urine discharge: (1) bladder muscles to squeeze urine out, (2) sphincter muscles to help the urethra open and close to allow urine flow, and (3) pelvic floor muscles to provide support for the bladder and urethra.

Urinary incontinence is a loss of “bladder control” and the inability to prevent the discharge of urine. Stress urinary incontinence (SUI) is the accidental leakage of urine during moments of physical activity (“stress”) that put pressure on the abdominal wall (lifting, coughing, sneezing, laughter, exercise). A significant majority of SUI patients are female, although the condition affects both men and women; it is an embarrassing condition that impacts quality of life of the patient.

SUI can be classified by severity (see Blaivas and Olsson 1988) and SUI etiology involves several factors associated with abnormalities or weakness in the sphincter, urethra, pelvic floor, anatomical positioning and movement, and other factors predominantly related to childbearing or obesity (Frost & Sullivan 2007). SUI is highly underreported and some estimates are that only 1 in 4 patients with SUI report the condition to their physician (Hampel, Wienhold et al. 1997). Numerous studies over the years point to “significant” SUI prevalence (see Bump and Norton 1998; see also Mark and, Richter et al. 2011 and Wu, Kawasaki et al. 2011); and the nature of SUI makes definitive statistics elusive due to differences in study population and definitions of SUI, with estimates varying that between 35%-69% of women are affected (Markland, Richter et al. 2011). An absence of longitudinal studies makes estimates of incidence very difficult, while prevalence statistics alone speak to a very large unmet and underreported medical need. SUI prevalence, impact on quality of life, and economic cost collectively represent a public health burden. It has been estimated that the incremental lifetime medical cost of SUI is up to $58,000 per woman.

SUI is considered treatable. Non-surgical methods are a first-line therapy, often involving a course of behavioral or pelvic floor muscle therapy and medication. Alternatives, such as continence pessaries, represent other conservative therapies. Despite the success of non-surgical methods, they are not always successful and often surgery is required, especially in cases where incontinence severity increases over time.

Over the past two decades, several different surgical procedures have emerged, with the most popular procedure involving the implantation of a pelvic sling (often described as a “hammock”) around the urethra (see Frost & Sullivan 2005). FIG. 1 shows such a sling 40 installed to support a bladder 10 and a Urethra 30 in a pelvic bone 20. Essentially, the pelvic sling provides additional anatomic support of the urethra to improve urethral resistance—thus preventing leakage—while at the same time preserving voluntary bladder voiding. It is useful to distinguish between the different procedures, sling positions, sling materials, and sling designs, since efficacy and morbidity varies amongst them (see Frost & Sullivan 2007; Daneshgari, Kong et al. 2008).

Although considerable attention has been made to immediate and long-term post-surgical complications (e.g., inability to urinate, problems emptying the bladder, and sudden urge to urinate), sling surgery has a high patient satisfaction rate, and hence remains a preferred therapeutic option for many women (see Frost & Sullivan 2005). Mid-urethral vaginal slings are the mainstay surgical treatment, totaling approximately 165,000 procedures annually.

One source of post-surgical complications is believed to be in the variance in sling tension—if the sling is placed too tightly (excessive tension), there may result a blockage in the bladder outlet. Too loosely (insufficient tension) reduced the effectiveness of the sling. Despite advancements in sling technology, tension adjustment during sling placement remains a non-standardized variable that is subject to surgeon's personal judgment.

### SUMMARY

Provided are a plurality of example embodiments, including, but not limited to, a handheld pressure probe that surgeons can use intraoperatively to measure pressure applied by mid-urethral slings on the urethra to address stress urinary incontinence. A smart probe is provided for use during an implantation procedure to provide a desired spacing between the implanted device and a body part while measuring the pressure exerted by the implanted device on the body part to ensure that the implanted device incorporates a desired tension, and thereby a desired pressure, on the body part. Also, a procedure for using the smart probe during the implantation procedure is disclosed.

Further provided is a “smart spacer” that can measure pressure and alert the surgeon if the pressure is above a certain limit.

Further provided is the above probe device that is disposable.

Also provided is a handheld probe system which enables a surgeon to measure mesh pressure and compare against a calibrated scale to estimate placement relative to a standard.

Also provided is a method of measuring and recording changes in pressure on the vaginal sling over time, such that the pressure can be recorded and used to compare such pressure measurements against clinical outcomes.

Further provided is a method of using the example system for deployment/use of example probe devices for pressure sensing/measurement for use in drawing intelligent information from those measurements over time.

Also provided is a method of collecting and displaying pressure between the mesh and urethra measured by any of the example systems.

Further provided is a method of calibrating any of the example systems.

Still further provided is a method of calibration using a calibration scale to effect a known delta-X that provides an expected delta-P.

Further provided is a medical probe for detecting a pressure imposed on a body part by a medical implant, the probe comprising: a probe body configured to provide a desired diameter to set a desired pressure of the medical implant on the body part during implantation of the medial implant; a pressure sensor installed in the probe body, the senor configured to provide a pressure signal indicating a sensed implant pressure imposed on the body party by the medical implant; a display; and electronics configured to convert the pressure signal into a display signal provided to the display configured to display an indication of the sensed implant pressure.

Also provided is a medical probe for detecting a pressure imposed on a body part by a medical implant, the probe comprising: a probe body configured to provide an adjustable diameter to set a desired pressure of the medical implant on the body part during implantation of the medial implant; a pressure sensor installed in the probe body, the senor configured to provide a pressure signal indicating a sensed implant pressure imposed on the body party by the medical implant; a display configured to display a numerical value; and a controller configured to convert the pressure signal into a display signal provided to the display configured to display a numerical indication of the sensed implant pressure.

Further provided are any of the above devices wherein the display is further configured to indicate when the desired pressure is achieved.

Also provided are any of the above devices further comprising a transmitter configured to transmit the sensed implant pressure to an external device, and/or wherein the sensor is integrated into the probe body, and/or wherein the controller and display are configured to be removable from the probe for reuse such that the probe body with the sensor are configured to be disposed after use.

Also provided are any of the above devices wherein the probe body is configured to provide the adjustable diameter using a pivot such that a gap provided at one end of the probe body is adjustable by pivoting two different parts of the probe body about the pivot, which may also further comprise a mechanism for biasing the gap to automatically increase or decrease the gap.

Further provided is a method of implanting a medical sling in a patient, comprising the steps of: (1) providing a medical sling configured to provide pressure on a body part of the patient when implanted in the patient; (2) providing a probe (such as one described herein) configured to provide a desired diameter and also configured to measure a pressure for display; (3) at least partially implanting the sling in the patient; (4) inserting the probe providing the desired diameter between the sling and the body part of the patient to tension the sling; (5) measuring the pressure between the sling and the body part using the probe; (6) adjusting, if necessary, the tension in the sling to provide a desired pressure between the sling and the body part; and (7) when the desired pressure between the sling and the body part has been obtained, completing the implantation of the sling in the patient.

Also provided are additional example embodiments, some, but not all of which, are described herein below in more detail.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 shows a line drawing of an example vaginal sling implanted in a patient;

FIG. 2 shows a plurality of differently sized dilators for use in the medical procedure for implanting a sling;

FIG. 3 shows a drawing of an example dilator adapted for measuring sling pressure;

FIG. 4 shows a schematic diagram of the example dilator of FIG. 3;

FIG. 5 shows a schematic diagram of another example dilator having wireless capability;

FIG. 6 shows a block diagram of circuitry for the example dilator of FIG. 4;

FIG. 7 shows a block diagram of circuitry for the example dilator of FIG. 5;

FIG. 8 is diagram showing a perspective view of an example adjustable dilator adapted for measuring sling pressure; and

FIG. 9 is a side view of the example dilator of FIG. 8.

### DETAILED DESCRIPTION OF THE EXAMPLE EMBODIMENTS

This application describes the design and application of examples of a number of different versions of a disposable or sterilizable and reusable “pressure sensitive probe” (or dilator) that will allow a surgeon to surgically place a pelvic (vaginal) sling tuned at specific desired amounts of tension to provide a desired pressure on the patient's urethra.

The example processes using example smart probe/dilator devices described herein are provided with the capability to measure sling pressure against the urethra or bladder during placement of a mid-urethral vaginal sling in a surgical procedure, where the patient is provided in a supine position as the sling/mesh is installed. Since such installation conditions do not represent the expected tensions that will occur when the patient is standing or sitting, a dilator/spacer is used between the sling/mesh and the urethra, bladder, or other body part to adjust the tension of the sling/mesh to a desired predetermined value while it is being installed/implanted to approximate the desired non-supine tension. Standard practice would include use of FDA approved devices that have undergone efficacy clinical trials for such a product in the healthcare market. Providing a smart probe as disclosed herein to measure and notify the surgeon of the measured pressure provides an advancement in the art.

There are three main categories of slings that can be utilized in the covered procedures: (a) tension-free (b) adjustable, and (c) conventional. Although the present probe/dilator system is associated with conventional sling clinical procedures, the pressure measurement and calibration system described herein has applicability for all sling categories.

The smart probe system can utilize non-disposable and disposable components, as desired. Disposable components would be assigned a HCPCS code, much in the same way the sling mesh is a consumable and has a HCPCS code.

In clinical practice, the smart dilator system (smart probe) would be used to standardize sling placement over current more variable practice. There is potential for wide use of the system, which could benefit over 165,000 women who annually undergo this procedure. A secondary market includes men with stress urinary incontinence, a possible consequence from prostate surgery, and desire a male sling.

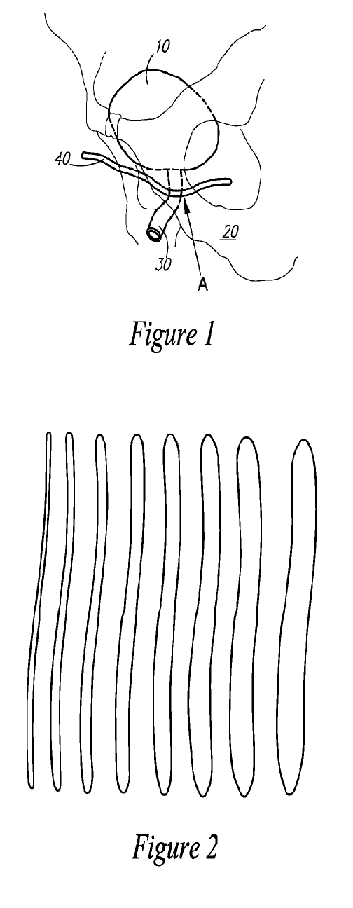

In clinical practice, such as shown in FIG. 1, a synthetic mesh/sling 40 is used as a “hammock” against the pubourethral ligament, compressing the lumen of the urethra 30 to prevent involuntary voiding during increased intra-abdominal pressure (e.g. coughing and sneezing). When a mid-urethral vaginal sling is surgically placed, there have in the past been no set parameters on how tight to pull the sling (i.e., its tension), which could contribute to urinary retention, voiding dysfunction or new onset urge incontinence secondary to partial bladder outlet obstruction. Currently, the most common technique is to place an instrument of a standard size between the urethra and sling in an attempt to ensure consistency; however, when placing the sling into position the amount of tension placed on the sling can vary between surgeons and sling designs. The proposed surgical probe system offers the opportunity to:
Reduce complication rates;
Establish objective standards to supplement sling procedure;
Reduce the variance in sling tension and pressure for any given procedure;
Improve “quality of life”; and
Improve patient willingness to seek treatment.

The present disclosed example solutions relate to systems, devices and methods for sensing, analyzing, and reporting on one or more physiologic and physical parameters associated with the SUI mesh implants, such that the individual clinician is provided with an estimate of their current bodily function.

Complications associated with SUI mesh implants can potentially be mitigated if measurement systems to quantify mesh tension and urethral pressure are made available. Current practice involves extensive training and experience to develop tacit and tactile knowledge that experts acquire over time. The proposed example improvements using the smart probe design, as discussed herein, bring to the aid of the surgeon handheld measurement tools that can be calibrated to surgical set-points and lead to more objective implant standards. Surgeons who are experts in this field have collaborated to develop the proposed hand-held instruments described in this disclosure that can meet the objective measurement needs of SUI mesh implants and thereby simplify the procedure and improve efficacy. In general terms, the example solutions and their significance are that:
A novel handheld probe system enables a surgeon to measure mesh pressure and compare against a calibrated scale to estimate placement relative to a standard;
Probe data can be collected and displayed for pressure between the mesh and urethra; this critical information is currently unavailable in typical patient settings;
Even in the tension-free situation, some of the example probe systems feature expandable sizing set-points that can simulate calibrated “over-pressure” situations critical to proper mesh placement when mesh tension would normally be absent; and
A variety of SUI mesh types to be accommodated by the proposed system and approach.

The accompanying figures depict multiple embodiments of the system and device for sensing, analyzing, and reporting on one or more physiologic and physical parameters associated with the SUI mesh implants. A more detailed description of parts of the subsystem and relevant figures is provided below. Those of ordinary skill in the art can readily use these examples to provide alternative embodiments using the ideas and teaching contained herein.

An embodiment of a smart probe spacer system development features an integrated probe-data collection system which includes the ability to directly measure pressure between the mesh and the urethra, or to measure that pressure provided by a fixed or an expandable sizer placed between the mesh and the urethra, for which a given expansion above a nominal value will enable calibration of a mesh implant that has little or no bias pressure present. Example embodiments of the smart probe are discussed in detail.

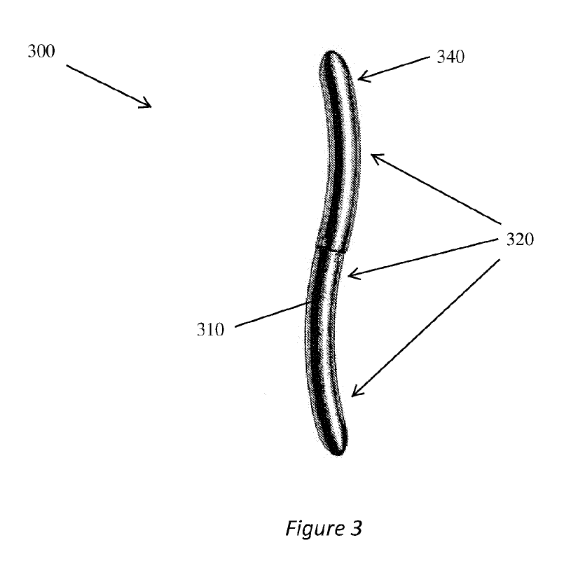

FIG. 2 shows a plurality of fixed diameter dilators (spacers), each of which can be configured to incorporate components for measuring a pressure imposed by a mesh/sling during its installation/implantation in medical procedures as described herein. FIG. 3 shows an example of one such pressure measuring dilator device 300 having a body 310 of fixed diameter that is configured to have a display 340 for showing a tension or acceptable tension indicator, a sensor 320 along the length of the dilator. Note that although “S” shaped curved fixed dilators are shown, embodiments that have different shapes of curves, or that are straight, could also be provided.

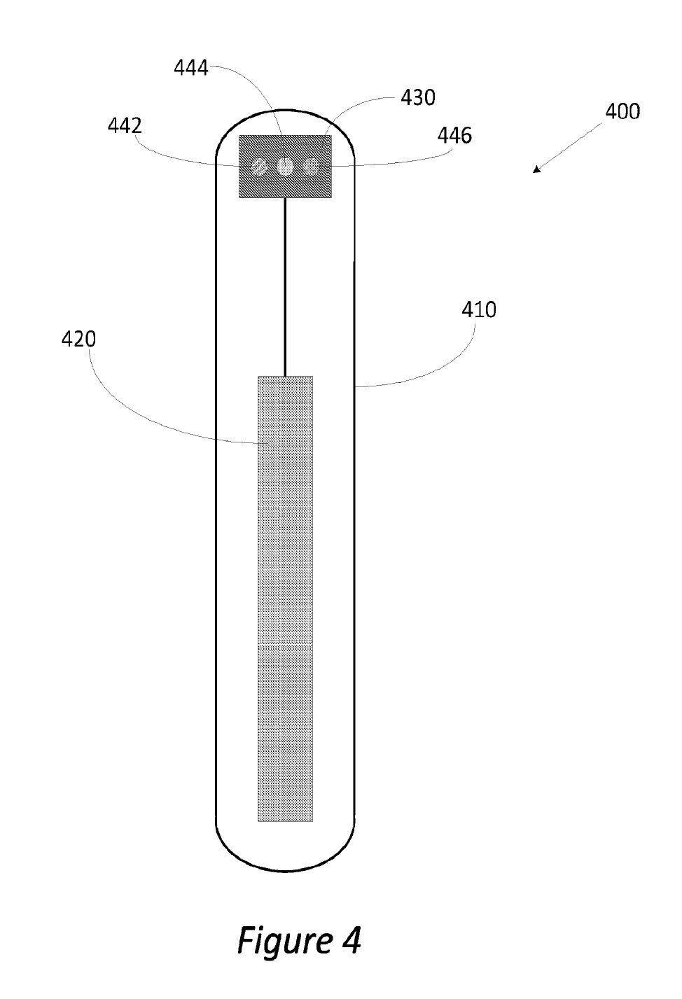

FIG. 4 is a schematic drawing of such an example fixed diameter smart dilator (spacer) device 400 (with body curvature removed for clarity) having a body 410 with a sensor 420 provided along some portion of its length (which can vary based on the particular application of the dilator). A control or processor or other electronics unit 430 may be provided that receives the information from a pressure sensor 420 to process or otherwise determine whether the pressure detected by the sensor 420 falls within a desired range. Display lights 442, 444, and 446 (e.g., LED indicators) can be configured to provide indications as to whether the pressure is acceptable (e.g., green light), near an acceptable range (e.g., yellow light), or is outside of an acceptable range (e.g., red light). The pressure ranges may be predetermined based on clinical trials, and/or may be resettable based on lessons learned, changing procedures, or to support particular applications.

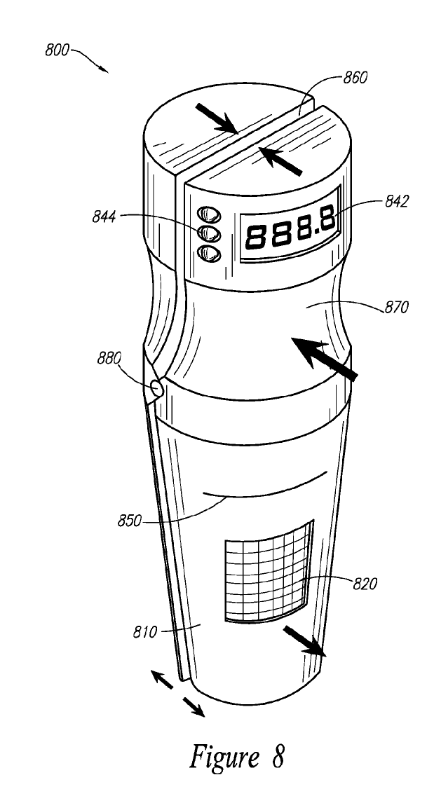

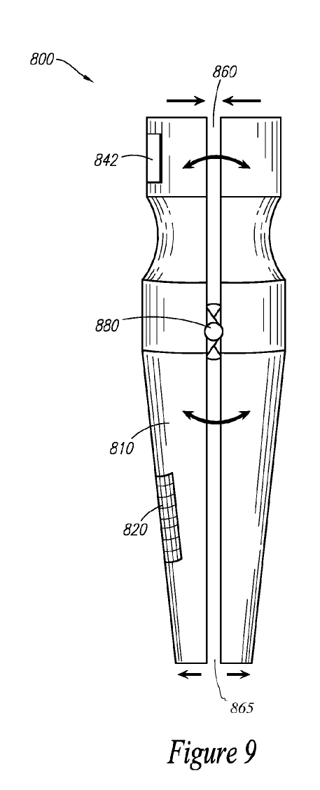

In contrast to providing the three indicators 442, 444, and 446, a single multi-colored indicator (e.g., multi-colored LED) may be used to indicate the different pressure ranges, with other indicators being provided to show that the unit is active (i.e., “on” or “off”), and/or to show that the battery is charged (or nearly depleted), for example. Alternatively, a display that shows a numeric indication of the actual measured pressure could be used (such as described for the embodiment of FIGS. 8-9, below). Or combinations of these displays could be used, as desired, such as to both show a numeric indication of measured pressure and also to show when a desired pressure has been reached.

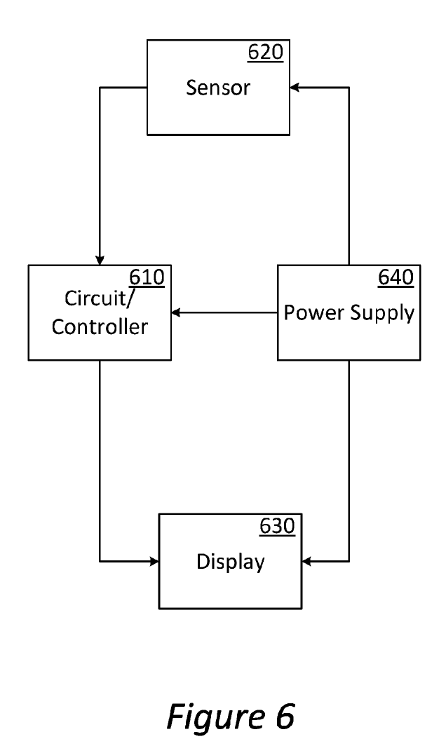

FIG. 6 shows a block diagram of example electronic components that could be used for the example smart dilator (spacer) of FIGS. 3-4. A pressure sensor 620 can be provided that senses the pressure between a sling being installed and the patient's urethra (or other body part), for example. Examples of sensor types that could be used for such a pressure sensor 620 include strain gauge, capacitive, and piezoelectric sensors, all of which are known in the art. Other types of pressure sensors could also be utilized.

The output signal of the pressure sensor would be provided into a circuit/controller 610 that converts the measured signal into control signals to provide a proper display indication on display 630. In its simplest form, the circuit could be a simple electronic circuit comprising basic electronic components include transistors and resistors, for example, that switches certain indicator lights (e.g., items 442, 444, and 446 of FIG. 4) “on” or “off” for certain ranges of sensor outputs based on sensor thresholds, for example. In more complicated designs, the circuit 610 would be a programmable controller (or a dedicated controller, such as an ASIC, using firmware, for example) that examines the sensor signal and determines the measured pressure and then generates the display data for the display 630 to show the pressure or status indication. A power supply 640 is provided to power the components, and may include a disposable or rechargeable battery. The display 630 could be a numeric LCD display, for example, with any needed associated display drivers being provided as part of the display 630 (or alternatively part of the circuit 610)

The electronic components might be comprised of off-the-shelf components that can be sterilized before and after use, or in the case of a disposable dilator, can be disposable for a one-use scenario. In some embodiments, some of the electronic components might be removable and reusable. For example, the sensor could be made integral with the dilator body which would be disposed after use, but the other electronic components (610, 630, 640) might be removable and reusable, since separating such electronics from the sensor enables a lower cost overall system of measurement as compared to a disposable device.

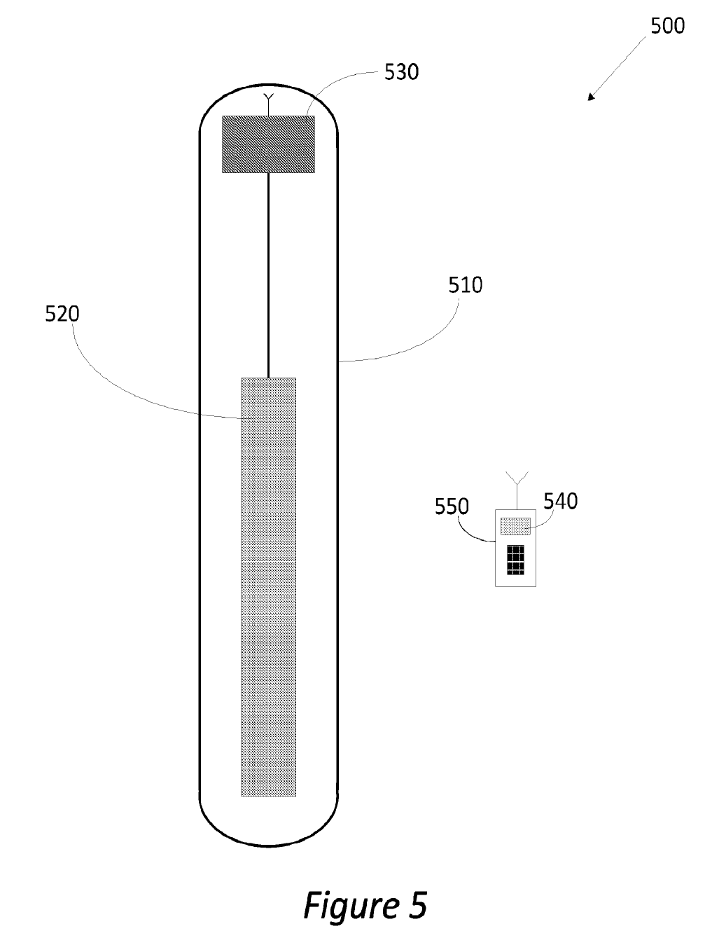

FIG. 5 shows an alternative system 500 that has a pressure detecting smart dilator (spacer) device 510 that is configured to communicate wirelessly with an external device 550 having a display 540 that can be located remotely. This device uses a pressure sensor 520 (such as described elsewhere herein, for example) to generate a pressure sensor signal, and includes electronic components 530 for processing the sensor signal and transmitting the result to the external device 550. This device may or may not have an integrated display.

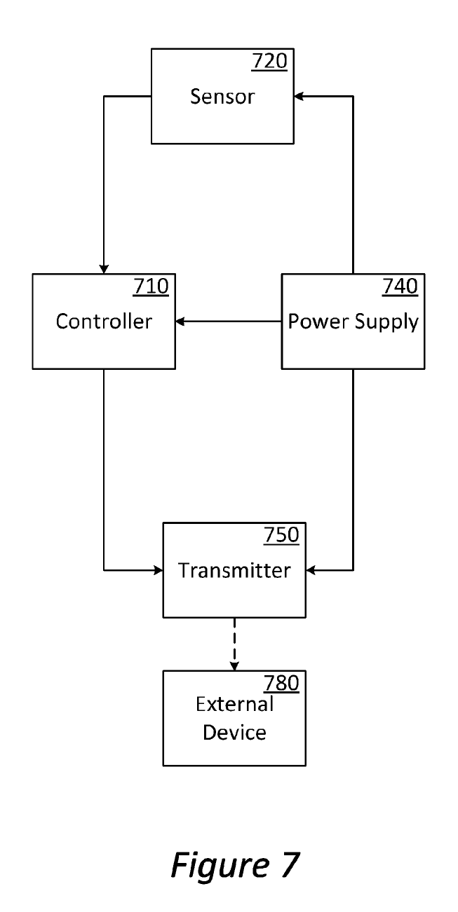

FIG. 7 shows example electronic components that may be used for such a wireless smart dilator device of FIG. 5. The pressure sensor 720 provides its pressure signal to a programmable controller 710, which processes the signal and provides information for transmitting to the transmitter 750, which communicates with the external (remote) device 780. Wireless communication could be by RF, WiFi, or Bluetooth protocols, for example. The wireless device 780 could be a tablet computer, smartphone, or some other device having display capability to display the sling/mesh pressure measured by the dilator device. The external device 780 might also utilized audio tones to show when the pressure is within, or outside of, an acceptable range.

As described above, the smart dilator device 510 may be fully reusable (i.e., sterilizable), fully disposable, or some components might be made removable for reuse (e.g., the electronic components) while the remainder (e.g., body with integrated sensor) may be made disposable, in which case the reusable components can be removed form a used device and installed in a new body for reuse.

Note that rather than using a plurality of smart probes having different diameters, one smart probe having one particular diameter might be used to provide an initial diameter, and then additional spacers/probes having different diameters might be used in addition to the smart probe to vary the total diameter and thereby vary the tension in the sling. In this manner, only one such probe/spacer need be configured to measure pressure, whereas additional spacers are used to further vary the tension in the sling during installation while using the original smart probe to continue to measure the pressure, until the desire pressure is achieved and the sling fixed in place.

FIGS. 8-9 show another example embodiment of a smart dilator (spacer) device 800 where the diameter is variable. The example embodiment of FIGS. 8-9 show a device body 810 having two halves split along an axis of its length, with a gap provided between them to provide an adjustable diameter by varying gaps 860, 865 (inversely) using a pivot/hinge 880 to vary the gaps. The pivot 880 may be accompanied by one or more springs or a compliant material (e.g., rubber) to allow the device to return to a resting state to set the gaps to an initial value. In particular, automatically spreading out the upper gap is beneficial, as the gap can be decreased by pressing on the portion 870.

The example device 800 has a numeric display 842 for displaying an actual measured pressure as obtained by pressure sensor 820 and processed by a programmable controller (not shown). Indicator lights 844 are provided for showing status of the device 800 (e.g., power state, battery state), and an indicator could also be used for showing when the pressure is in a predetermined acceptable range. Indicator 850, which may be a line or mark provided on the body of the device, is provided to show the appropriate “mesh line” for properly using the smart dilator for measuring the pressure of the mesh/sling during installation. The device diameter of the device is adjusted by pressing on portion 870, which rotates the device about the pivot 880 to increase or decrease the effective diameter of the device thereby increasing or decreasing the tension on the sling.

Because the device 800 is adjustable, it could be more cost effective than using the fixed diameter probes by not requiring the manufacture of differently sized dilators. Instead, the device is adjusted at the sling/mesh tension is varied to obtain the optimal desired amount during installation of the sling. Note that rather than, or in addition to, having a numerical display, this device could have display indicators similar to the device of FIG. 4, or it could utilize a remote display device as in the embodiment of FIG. 5, or some combination of these features.

Another alternative embodiment of the probe system development has an integrated probe-data collection system that incorporates a calibration system to enable the ability to directly measure pressure between the mesh and the urethra in response to actuated standard “over-pressures” provided by a calibrated expandable sizer or balloon placed between the mesh and the urethra for which a given expansion above a nominal value will enable pressure between a mesh and urethra.

Additional example probe devices can be used to detect a change in pressure on the vaginal sling even after installation. Over time, the pressure can be recorded and used to compare such pressure measurements against clinical outcomes. One novel aspect in the deployment/use of such probe devices is for pressure sensing/measurement for use in drawing intelligent information from those measurements over time.

At the time of vaginal mesh deployment, the surgeons use a “spacer” (traditionally a passive dilator device) between the mesh and the urethra to prevent the mesh directly tensioning the urethra. The improved devices disclosed herein are actually “smart spacers” that can measure pressure and alert the surgeon if the pressure is above a certain limit. Such pressure ranges might be between 0.25 oz and 0.40 oz, for example.

The ability of the surgeon to monitor pressure in real-time is central to achieving optimal sling placement and optimal procedure outcomes.

Optionally, stress/pressure responsive colored mesh material can be used to indicate that if the surgeon goes above a certain pressure/stress limit-its color will change signifying violation of the limit. Or rather than using a dilator that measures pressure electrically, a mechanically based system could be used that changes color based on a mechanically detected pressure to provide a purely mechanical option that does not require electronic components.

### APPLICATIONS

A clinical challenge in sling placement is that the lithotomy position is most appropriate patient position for conducting the surgical procedure; unfortunately, this is not the same standing or sitting position (orthostatic position) the patient assumes for activities of daily living where SUI is an issue. In the lithotomy position the patient is lying supine with legs slightly elevated relieving pressure on the pelvic floor due to the action of gravity on the bladder and other organs. In the orthostatic position gravity restores pressure on organs.

During the sling placement procedure there is the need to estimate how the sling should be placed in the lithotomy position such that the “correct” (desired) pressure exists when the patient stands (orthostatic position, where gravity acts on the bladder and other organs). The current standard of care does not offer a way for the surgeon to objectively determine the grams of pressure to be exerted on the spacer used to place the sling during sling deployment/placement—clinically

During sling installation, one can effect an upward force generated with sling pull and counter resistance by the surgeon who is handling a spacer between the mesh and the urethra. This dynamic pull/resistant process is captured by placing the sling in the lithotomy position and should result in a no-tension placement while the patient is in lithotomy position.

The smart spacer, such as an embodiment disclosed herein, allows the surgeon to use a calibrated scale to temporarily provide a desired slight actual tension in the mesh, by measuring the pressure between the sling/mesh and a body part, to provide for the lithotomy position that correlates to satisfactory clinical outcomes when the patient returns to orthostatic position post-operatively.

Specifically, to perform one example procedure using the device to installing a vaginal sling for treating incontinence, a 16 French urethral catheter is inserted and connected to drainage. In the dorsal lithotomy, the anterior vaginal wall at the level of the mid-urethra is exposed with the use of a weighted vaginal retractor. The anterior vagina is hydro-dissected with either injectable saline injection or injection of lidocaine with epinephrine at the level of the mid-urethra. Then, a 1 cm incision is made in the anterior vaginal mucosa 1 cm from the urethral meatus and 1.5 cm long. The vaginal mucosa is then dissected away from the underlying urethra laterally creating two vaginal tunnels to both the right and left side of the urethra.

For an up-down retro pubic sling, two stab incisions are made in the suprapubic area 1.5 cm lateral to the midline. The metallic trocars of the sling (manufacturer specific) is inserted into the stab incision, perforating rectus fascia, hugging the posterior aspect of the symphysis, perforating endopelvic fascia and exciting into the vaginal tunnel that were created. This is done on both the right the left side. A cystoscopy (looking inside the bladder) is done to ensure no bladder injury with passage of the trocars. Then the sling (typically encased in a plastic sheath) is attached to the trocars and the trocars are pulled back pulling the arms of the sling into the retro pubic space and through the rectus muscle and out of the stab incisions. Typically, a size 9 hegar dilator is placed between the urethra and the sling (still within the plastic sheath) as the sling arms and the trocars are being pulled. Hence, the smart probe is placed between the sling and the urethra during the dynamic placement of the sling, and is removed when the plastic sheets are removed and sling is deployed.

It is the dynamic pull of the sheath that actually deploys the sling around the urethra, and this is the only step of the sling placement that is not standardized. During the pull of the sheath, the operating surgeon holds the Hegar dilator (in this case using the smart probe) and exerts a steady resistance against the pull to prevent sling over tensioning. The smart probe, such as in the example embodiments provided herein, allows the surgeon to measure the pressure that the smart Hegar dilator probe “feels” during this dynamic process, and the probe displays digitally the measured pressure, for example. Eventually for each sling approach (up-down retro pubic-described above, down-up retro pubic, in-out tranobtrurator, out-in trans-obturator or any iteration of mini-slings), the average pressure that is associated with ideal outcome would be determined, and surgeon would be able to control the pull and the resistance balance to keep the grams within the safe zone for ideal outcome, in which case the implanted sling has been provided with the desired tension to properly interact with the urethra. Ideal outcome balances optimizing efficacy and lower obstruction.

Many other example embodiments can be provided through various combinations of the above described features. Although the embodiments described hereinabove use specific examples and alternatives, it will be understood by those skilled in the art that various additional alternatives may be used and equivalents may be substituted for elements and/or steps described herein, without necessarily deviating from the intended scope of the application. Modifications may be necessary to adapt the embodiments to a particular situation or to particular needs without departing from the intended scope of the application. It is intended that the application not be limited to the particular example implementations and example embodiments described herein, but that the claims be given their broadest reasonable interpretation to cover all novel and non-obvious embodiments, literal or equivalent, disclosed or not, covered thereby.

1. A medical probe for detecting a pressure imposed on a body part by a medical implant, said probe comprising:
a probe body configured to provide a desired diameter to set a desired pressure of the medical implant on the body part during implantation of the medial implant;
a pressure sensor installed in said probe body, said senor configured to provide a pressure signal indicating a sensed implant pressure imposed on the body party by the medical implant;
a display; and
electronics configured to convert the pressure signal into a display signal provided to the display configured to display an indication of the sensed implant pressure.

2. The medical probe of claim 1, wherein said probe body is configured to provide a fixed predetermined diameter.

3. The medical probe of claim 1, wherein said probe body is configured to provide an adjustable diameter that can be adjusted during the implant procedure to vary the pressure of the medical implant on the body part during implantation of the medial implant to obtain the desired pressure.

4. The medical probe of claim 1, wherein said electronics includes a programmable controller.

5. The medical probe of claim 1, wherein said display is configured to indicate when the desired pressure is achieved.

6. The medical probe of claim 1, wherein said display is configured to display a numerical indication of the sensed implant pressure.

7. The medical probe of claim 1, wherein said electronics are configured to transmit the sensed implant pressure to an external device.

8. The medical probe of claim 1, wherein said sensor is integrated into said probe body.

9. The medical probe of claim 8, wherein said electronics are configured to be removable from said probe for reuse, and wherein said probe body with said sensor is configured to be disposed after use.

10. The medical probe of claim 1, further comprising a rechargeable battery for providing power to said electronics and said display.

11. A medical probe for detecting a pressure imposed on a body part by a medical implant, said probe comprising:
a probe body configured to provide an adjustable diameter to set a desired pressure of the medical implant on the body part during implantation of the medial implant;
a pressure sensor installed in said probe body, said senor configured to provide a pressure signal indicating a sensed implant pressure imposed on the body party by the medical implant;
a display configured to display a numerical value; and
a controller configured to convert the pressure signal into a display signal provided to the display configured to display a numerical indication of the sensed implant pressure.

12. The medical probe of claim 11, wherein said display is further configured to indicate when the desired pressure is achieved.

13. The medical probe of claim 11, further comprising a transmitter configured to transmit the sensed implant pressure to an external device.

14-16. (canceled)

17. The medical probe of claim 11, wherein said probe body is configured to provide the adjustable diameter using a pivot such that a gap provided at one end of the probe body is adjustable by pivoting two different parts of said probe body about the pivot.

18. The medical probe of claim 17, further comprising a mechanism for biasing said gap to automatically increase or decrease the gap.

19. A method of implanting a medical sling in a patient, comprising the steps of:
providing a medical sling configured to provide pressure on a body part of the patient when implanted in the patient;
providing a probe configured to provide a desired diameter and also configured to measure a pressure for display;
at least partially implanting the sling in the patient;
inserting the probe providing the desired diameter between said sling and the body part of the patient to tension the sling;
measuring the pressure between the sling and the body part using the probe;
adjusting, if necessary, the tension in the sling to provide a desired pressure between the sling and the body part; and
when the desired pressure between the sling and the body part has been obtained, completing the implantation of the sling in the patient.

20. The method of claim 19, wherein the pressure measured by the probe is numerically displayed on a display.

21. The method of claim 20, wherein said display is on a device remote from said probe.

22. The method of claim 19, wherein said step of adjusting, if necessary, the tension in the sling, includes the step of varying the diameter of the probe.

23. The method of claim 19, wherein said step of adjusting, if necessary, the tension in the sling, includes the step of substituting or supplementing the probe having one fixed diameter with another probe having a different diameter.
